(19) United States
(12) Patent Application Publication
(10) Pub. No.: US 2007/0154894 A1
(43) Pub. Date: Jul. 5, 2007

(54) Labeling and non-enzymatic fragmentation of cDNA using a ribonucleoside triphosphate analog

(75) Inventors: Glenn H. McGall, Palo Alto, CA (US); Handong Li, San Jose, CA (US); Anthony D. Barone, San Jose, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/323,068

(22) Filed: Dec. 30, 2005

(51) Int. Cl. C12Q 1/68; C12P 19/34; C07H 21/04

(52) U.S. Cl. 435/6; 435/91.2; 536/25.32

(57) ABSTRACT

In accordance with the present invention, methods are presented for labeling a cDNA strand with a labeled ribonucleotide base precursor which upon exposure to Mg2+, heat and base cleaves the cDNA at each place of incorporation of an RNA. In accordance with an aspect of the present invention, compounds selected from the group consisting of 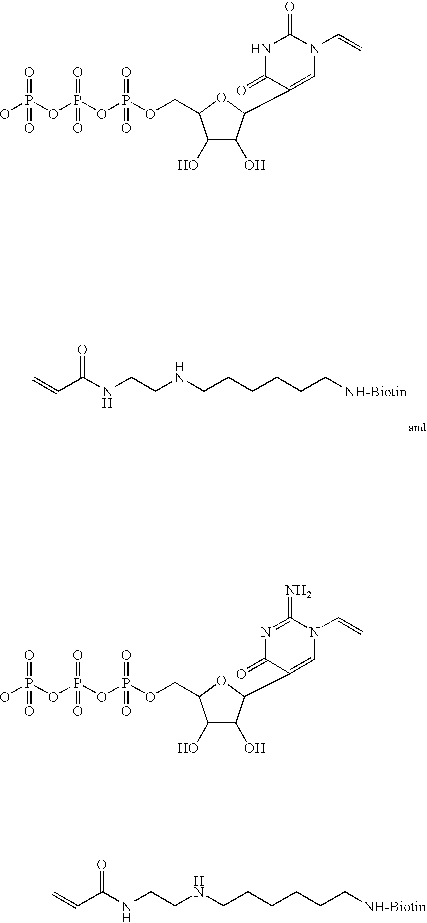
are incorporated into the growing strand of a cDNA by a reverse transcriptase or a mutant reverse transcriptase. After subject the strands to Mg²⁺, base and heat, the 3′ OH causes cleavage of the cDNA leaving a 2′OH phosphate with a biotin label. The biotin provides a label which may be bound to streptavidin and thereafter hybridized to a nucleic acid array.

### FIELD OF THE INVENTION

This invention relates generally to methods of preparation of nucleic acids for hybridization to a nucleic acid array. More particularly this invention relates to non-enzymatic methods for fragmentation of cDNA using ribonucleoside triphosphate analogs incorporated into cDNA chains.

### BACKGROUND OF THE INVENTION

Nucleic acid sample preparation and labeling methods have radically transformed laboratory research in the disciplines of genetics, molecular biology and recombinant DNA technology. Also impacted are fields as diverse as medical diagnostics, forensics, nucleic acid analysis and gene expression monitoring, to name a few. There remains a need in the art for methods for reproducibly and efficiently fragmenting and labeling nucleic acids used for hybridization on oligonucleotide arrays.

### SUMMARY OF THE INVENTION

Methods are provided for incorporating a labeled RNA nucleotide triphosphate into a cDNA to provide sites for transesterification and cleavage of the cDNA into labeled fragments which can be hybridized to an oligonucleotide array or a high density oligonucleotide array. In particularly preferred embodiments of the present invention the labeled RNA nucleotide triphosphates have the structure:

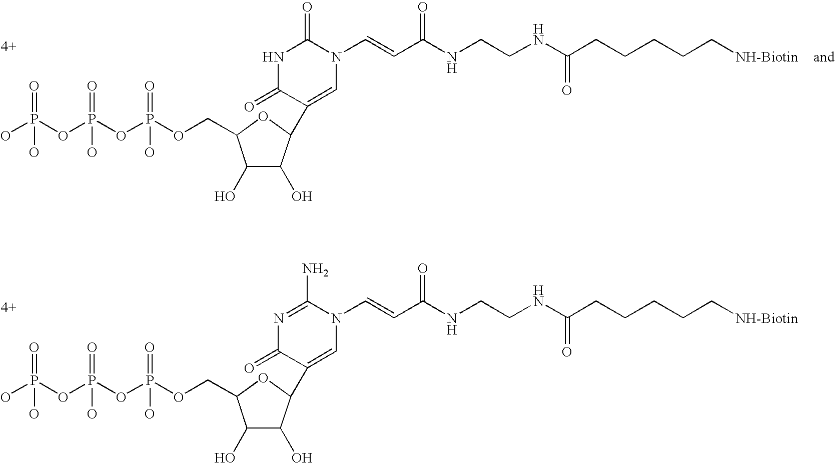

Alternative embodiments of the present invention are also presented wherein the RNA is not labeled, but is still used to specifically cleave the cDNA by either transesterification or treatment with a ribonuclease. These fragments are preferably labeled by the Biotin LTLS labeling system. Labeled fragments are then applied to a nucleic acid array for hybridization analysis.

In yet another embodiment of the present invention labeled deoxribonucleotides are incorporated into the cDNA. Fragments are produced with DNAse I and hybridized to a nucleic acid array.

### DETAILED DESCRIPTION OF THE INVENTION

A, General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” t includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, Biochemistry, (W H Freeman), Gait, “Oligonucleotide Synthesis: A Practical Approach” 1984, IRL Press, London, all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US 01/04285, and in U.S. patent applications Ser. Nos. 09/501,099 and 09/122,216 which are all incorporated herein by reference in their entirety for all purposes. Preferred arrays are commercially available from Affymetrix, Inc. (Santa Clara, Calif.). See www.affymetrix.com.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefor are shown in U.S. Ser. No. 10/013,598, and U.S. Pat. Nos. 5,856,092, 6,300,063, 5,858,659, 6,284,460 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. For example, see the patents in the gene expression, profiling, genotyping and other use patents above, as well as U.S. Ser. No. 09/854,317, Wu and Wallace, Genomics 4, 560 (1989), Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), Guatelli et al., Proc. Nat. Acad. Sci. USA, 87, 1874 (1990), WO 88/10315, WO 90/06995, and U.S. Pat. No. 6,361,947.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625 and in PCT Application PCT/US99/06097 (published as WO99/47964), each of which also is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over the internet. See provisional application 60/349,546.

B. Definitions

An “array of oligonucleotides or polynucleotides” also called “a library” as used herein refers to a multiplicity of different (sequence) oligonucleotides or polynucleotides attached (preferably through a single terminal covalent bond) to one or more solid supports where, when there is a multiplicity of supports, each support bears a multiplicity of oligonucleotides or polynucleotides. The term “array” can refer to the entire collection of oligonucleotides or polynucleotides on the support(s) or to a subset thereof. The term “same array” when used to refer to two or more arrays is used to mean arrays that have substantially the same oligonucleotide species thereon in substantially the same abundances. The spatial distribution of the oligonucleotide or polynucleotide species may differ between the two arrays, but, in a preferred embodiment, it is substantially the same. It is recognized that even where two arrays are designed and synthesized to be identical there are variations in the abundance, composition, and distribution of oligonucleotide or polynucleotide probes. These variations are preferably insubstantial and/or compensated for by the use of controls as described herein. The terms oligonucleotide and polynucleotide can be used interchangeably in this application and the use of one term should not appear as a limitation of the invention.

The term “biotin” as used in the context of an aspect of the present invention generally refers to the moiety represented by the following formula: 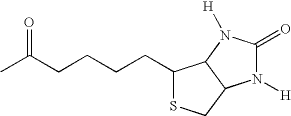
Molecules are generally shown in amide linkage to the biotin. Thus, for example, the nomenclature R—NH-biotin has the structure

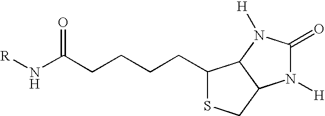

The terms “nucleic acid” or “nucleic acid molecule” as used herein refer to a deoxyribonucleotide or ribonucleotide polymer in either single or double stranded form. These terms also encompass DNA-RNA hybrids. Unless otherwise limited the phrase would also cover synthetic and naturally occurring variants of nucleic acids, including without limitation, base variants such as 7-deazapurine, 8-aza-7-deazapurine, isocytosine, pseudo isocytosine, and isouracil.

An “oligonucleotide” as used herein generally refers to a synthetic 2′-deoxynucleic acid ranging in length from 2 to about 200 nucleotides. An oligonucleotide may be double stranded or single stranded, but is more typically single stranded.

A “polynucleotide” as used herein refers to a single stranded or double stranded continuous nucleic acid of virtually unlimited length, i.e., a chromosome or circular plasmid might be referred to as a polynucleotide.

The term “primer” as used herein refers to a single-stranded oligonucleotide capable of acting as a point of initiation for template-directed nucleic acid synthesis under suitable conditions, for example buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with the template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified. A primer may include non-hybridizing sequences such as a transcription promoter.

As used herein a “probe” is defined as a nucleic acid capable of binding to a target nucleic acid of complementary sequence through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages. See U.S. Pat. No. 6,582,908. In the context of an array of nucleic acids, the “primer” is attached to the surface of the array, generally by covalent bonding.

The term “target nucleic acid” as used herein refers to a nucleic acid (often derived from a biological sample and hence referred to also as a sample nucleic acid), to which the oligonucleotide or polynucleotide probe specifically hybridizes. It is recognized that the target nucleic acids can be derived from essentially any source of nucleic acids (e.g., including, but not limited to chemical syntheses, amplification reactions, forensic samples, etc.). It is either the presence or absence of one or more target nucleic acids that is detected, or the amount of one or more target nucleic acids that is to be quantified. The target nucleic acid(s) that are detected preferentially have nucleotide sequences that are complementary to the nucleic acid sequences of the corresponding probe(s) to which they specifically bind (hybridize). The term target nucleic acid may refer to the specific subsequence of a larger nucleic acid to which the probe specifically hybridizes, or to the overall sequence (e.g., gene or mRNA) whose abundance (concentration) and/or expression level it is desired to detect. The difference in usage will be apparent from context.

The phrase “coupled to a support” means bound directly or indirectly thereto including attachment by covalent binding, hydrogen bonding, ionic interaction, hydrophobic interaction, or otherwise.

The term “detectable moiety” (Q) means a chemical group that provides a signal. The signal is detectable by any suitable means, including spectroscopic, photochemical, biochemical, immunochemical, electrical, optical, chemical, or radiological means. In certain cases, the signal is detectable by 2 or more means.

The detectable moiety provides the signal either directly or indirectly. A direct signal is produced where the labeling group spontaneously emits a signal, or generates a signal upon the introduction of a suitable stimulus. Radiolabels, such as ³H, ¹²⁵I, ³⁵S, ¹⁴C or ³²P, and magnetic particles, such as Dynabeads™, are nonlimiting examples of groups that directly and spontaneously provide a signal. Labeling groups that directly provide a signal in the presence of a stimulus include the following nonlimiting examples: colloidal gold (40-80 nm diameter), which scatters green light with high efficiency; fluorescent labels, such as fluorescein, Texas red, Rhoda mine, and green fluorescent protein (Molecular Probes, Eugene, Oreg.), which absorb and subsequently emit light; chemiluminescent or bioluminescent labels, such as luminol, lophine, acridine salts and luciferins, which are electronically excited as the result of a chemical or biological reaction and subsequently emit light; spin labels, such as vanadium, copper, iron, manganese and nitroxide free radicals, which are detected by electron spin resonance (ESR) spectroscopy; dyes, such as quinoline dyes, triarylmethane dyes and acridine dyes, which absorb specific wavelengths of light; and colored glass or plastic (e.g., polystyrene, polypropylene, latex, etc.) beads. See U.S. Pat. Nos. 3,817,837; 3,850,752; 3,939,350; 3,996,345; 4,277,437; 4,275,149 and 4,366,241.

A detectable moiety provides an indirect signal where it interacts with a second compound that spontaneously emits a signal, or generates a signal upon the introduction of a suitable stimulus. Biotin, for example, produces a signal by forming a conjugate with streptavidin having attached fluorescent labels, which are then detected. See Hybridization With Nucleic Acid Probes. In Laboratory Techniques in Biochemistry and Molecular Biology; Tijssen, P., Ed.; Elsevier: New York, 1993; Vol. 24. Biotin-streptavidin provides a particularly high level of signal as streptavidin can be fabricated to have a multiplicity of fluorescent labels.

A preferred detectable moiety is a fluorescent group. Fluorescent groups typically produce a high signal to noise ratio, thereby providing increased resolution and sensitivity in a detection procedure. Preferably, the fluorescent group absorbs light with a wavelength above about 300 nm, more preferably above about 350 nm, and most preferably above about 400 nm. The wavelength of the light emitted by the fluorescent group is preferably above about 310 nm, more preferably above about 360 nm, and most preferably above about 410 nm.

The fluorescent detectable moiety is selected from a variety of structural classes, including the following nonlimiting examples: 1- and 2-aminonaphthalene, p,p′diaminostilbenes, pyrenes, quatemary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

A number of fluorescent compounds are suitable for incorporation into the present invention. Nonlimiting examples of such compounds include the following: dansyl chloride; fluoresceins, such as 3,6-dihydroxy-9-phenylxanthhydrol; rhodamineisothiocyanate; N-phenyl-1-amino-8-sulfonatonaphthalene; N-phenyl-2-amino-6-sulfonatonaphthanlene; 4-acetamido-4-isothiocyanatostilbene-2,2′-disulfonic acid; pyrene-3-sulfonic acid; 2-toluidinonapththalene-6-sulfonate; N-phenyl, N-methyl 2-aminonaphthalene-6-sulfonate; ethidium bromide; stebrine; auromine-0,2-(9′-anthroyl)palmitate; dansyl phosphatidylethanolamin; N,N′-dioctadecyl oxacarbocycanine; N,N′-dihexyl oxacarbocyanine; merocyanine, 4-(3′-pyrenyl)butryate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis[2-(4-methyl-5-phenyl oxazolyl)]benzene; 6-dimethylamino-1,2-benzophenzin; retinol; bis(3′-aminopyridinium)-1,10-decandiyl diiodide; sulfonaphthylhydrazone of hellibrienin; chlorotetracycline; N-(7-dimethylamino-4-methyl-2-oxo-3-chromenyl)maleimide; N-[p-(2-benzimidazolyl)phenyl]maleimide; N-(4-fluoranthyl)maleimide; bis(homovanillic acid); resazarin; 4-chloro-7-nitro-2,1,3-benzooxadizole; merocyanine 540; resorufin; rose bengal and 2,4-diphenyl-3(2H)-furanone. Preferably, the fluorescent detectable moiety is a fluorescein or rhodamine dye.

Another preferred detectable moiety is colloidal gold. The colloidal gold particle is typically 40 to 80 nm in diameter. The colloidal gold may be attached to a labeling compound in a variety of ways. In one embodiment, the linker moiety of the nucleic acid labeling compound terminates in a thiol group (—SH), and the thiol group is directly bound to colloidal gold through a dative bond. See Mirkin et al. Nature 1996, 382, 607-609. In another embodiment, it is attached indirectly, for instance through the interaction between colloidal gold conjugates of antibiotin and a biotinylated labeling compound. The detection of the gold labeled compound may be enhanced through the use of a silver enhancement method. See Danscher et al. J. Histotech 1993, 16, 201-207.

The term “effective amount” as used herein refers to an amount sufficient to induce a desired result.

The term “fragmentation” refers to the breaking of nucleic acid molecules into smaller nucleic acid fragments. In certain embodiments, the size of the fragments generated during fragmentation can be controlled such that the size of fragments is distributed about a certain predetermined nucleic acid length.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-helix polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization conditions” as used herein will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “isolated nucleic acid” as used herein means a nucleic acid species that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The “heterocyclic group” or moiety (H) is a cyclic moiety containing both carbon and a heteroatom. Nonlimiting examples of heterocyclic groups contemplated by the present invention are purines and pyrimidines as well as 4-aminopyrazolo[3,4-d]pyrimidine; pyrazolo[3,4-d]pyrimidine; 1,3-diazole (imidazole); 1,2,4-triazine-3-one; 1,2,4-tri azine-3,5-di one; and, 5-amino-1,2,4-triazine-3-one.

The “linker moiety” (L) according to the present invention is covalently bound to the heterocycle at one terminal position and to the detectable moiety (Q) at another terminal position. It is of a structure that is sterically and electronically suitable for incorporation into a nucleic acid. Nonlimiting examples of linker moieties comprise one or more amido alkyl groups, alkynyl alkyl groups, alkenyl alkyl groups, functionalized alkyl groups, alkoxyl groups, thio groups and amino alkyl groups.

The term “monomer” as used herein refers to any member of the set of nucleotides, including ribonucleotides and 2′ deoxyribonucleotides that can be joined together to form an oligo or nucleic acid. For DNA, the group of nucleotides includes the naturally occurring G, A, T, and C. For RNA, the group of nucleotides includes G, A, U, and C. Monomers also includes both synthetic and naturally occurring variants of the above monomer. At the base position for example momoners include without limitation nucleotides having the following bases: deazaguanosine, inosine, 7-deaza A and G, 7-deaza-8-aza A and G, iso-C, pseudo-iso-C and iso-U.

The term “mRNA,” sometimes referred to “mRNA transcripts” as used herein, includes, but is not limited to, pre-mRNA transcript(s), transcript processing intermediates, mature mRNA(s) ready for translation and transcripts of the gene or genes, or nucleic acids derived from the transcript processing of mRNA. Transcript processing may include splicing, editing and degradation variants.

As used herein, a nucleic acid derived from a mRNA transcript refers to a nucleic acid for whose synthesis the mRNA transcript or a subsequence thereof has ultimately served as a template. Thus, a cDNA reverse transcribed from a mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of a gene or genes, cDNA reverse transcribed from the mRNA, cRNA transcribed from the cDNA, DNA amplified from the genes, RNA transcribed from amplified DNA, and the like.

The term “nucleic acid array,” sometimes referred to as an “library” as used herein refers to a synthetically or biosynthetically prepared collection of nucleic acids attached to a substrate. Arrays may be used, inter alia, to screen for the presence or absence of a nucleic acid in a sample. Substrates are available in a wide variety of different formats (for example, libraries of cDNAs or libraries of oligos tethered to resin, glass or silicon beads, silica chips, silicon or other solid or semi-solid supports). Additionally, the term “array” is meant to include those libraries of nucleic acids which can be prepared by spotting nucleic acids of essentially any length (for example, from 1 to about 1000 nucleotide monomers in length) onto a substrate. It also includes other method of fabrication, including photolithography, ink jet printing, and various forms of resists which can be selectively removed to allow fabrication of desired arrays. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated or halogenated nucleotides and nucleotide analogs. The sequence of nucleotides may be interrupted by non-nucleotide components for example by nucleotide analogs that undergo non-traditional hybridization. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

A “high density oligonucleotide array” is an array having a very large amount of genetic information encoded thereon. For example the Affymetrix U133 2.0 microarray provides comprehensive coverage of the entire transcribed human genome, allowing for the analysis of the expression levels of over 47,000 transcripts and variants, including 38,500 well-characterized human genes. This microarray is comprised of more than 54,000 probe sets and 1,300,000 distinct oligonucleotide features. See www.affymetrix.com.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide,” sometimes referred to as a “polynucleotide” as used herein refers to a nucleic acid ranging from at least 2, preferable at least 8, and more preferably at least 20 to 25 nucleotides in length. In preferred embodiments of the present invention, polynucleotides range from hundreds to thousands of nucleotides (if single stranded) or base pairs (if double stranded). Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acids (PNAs). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

“Bind(s) substantially” refers to complementary hybridization between a probe nucleic acid and a target nucleic acid and embraces minor mismatches that can be accommodated by reducing the stringency of the hybridization media to achieve the desired detection of the target oligonucleotide or polynucleotide sequence.

The phrase “hybridizing specifically to”, refers to the binding, duplexing, or hybridizing of a molecule preferentially to a particular nucleotide sequence under stringent conditions when that sequence is present in a complex mixture (e.g., total cellular) DNA or RNA. The term “stringent conditions” refers to conditions under which a probe will hybridize preferentially to its target subsequence, and to a lesser extent to, or not at all to, other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The terms “background” or “background signal intensity” refer to hybridization signals resulting from non-specific binding, or other interactions, between the labeled target nucleic acids and components of the oligonucleotide or polynucleotide array (e.g., the oligonucleotide or polynucleotide probes, control probes, the array substrate, etc.). Background signals may also be produced by intrinsic fluorescence of the array components themselves. A single background signal can be calculated for the entire array, or a different background signal may be calculated for each region of the array. In a preferred embodiment, background is calculated as the average hybridization signal intensity for the lowest 1% to 10% of the probes in the array, or region of the array. In expression monitoring arrays (i.e., where probes are preselected to hybridize to specific nucleic acids (genes)), a different background signal may be calculated for each target nucleic acid. Where a different background signal is calculated for each target gene, the background signal is calculated for the lowest 1% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is of mammalian origin). Background can also be calculated as the average signal intensity produced by regions of the array that lack any probes at all.

The term “quantifying” when used in the context of quantifying nucleic acid abundances or concentrations (e.g., transcription levels of a gene) can refer to absolute or to relative quantification. Absolute quantification may be accomplished by inclusion of known concentration(s) of one or more target nucleic acids (e.g. control nucleic acids such as BioB or with known amounts the target nucleic acids themselves) and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

C. Non-Enzymatic and Enzymatic Fragmentation and Labeling

According to one aspect of the present invention, the hybridized nucleic acids are detected by detecting one or more labels attached to the sample nucleic acids. In a preferred embodiment, label is simultaneously incorporated during amplification of the target nucleic acids. According to one aspect of the present invention, the target is mRNA. The mRNA is reverse transcribed with a reverse transcriptase enzyme using DNA nucleotides G, A, T and C, for example. In addition reverse transcription is performed with a labeled ribonucleotide, e.g., 2, as shown below.

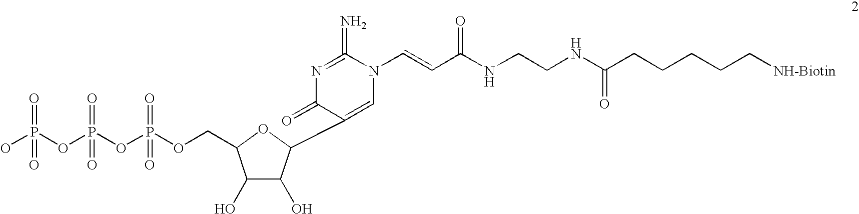

### Labeled Ribonucleotide Probe (Biotinylated ψ-iso-C)

By virtue of having an RNA nucleotide incorporated into a DNA strand, one can initiate a transesterification reaction, cleaving the cDNA product. By incorporating a labeled RNA as shown above, the cleaved fragments are each labeled. Alternatively, one could use a ribonuclease to cleave at each ribonuclease template.

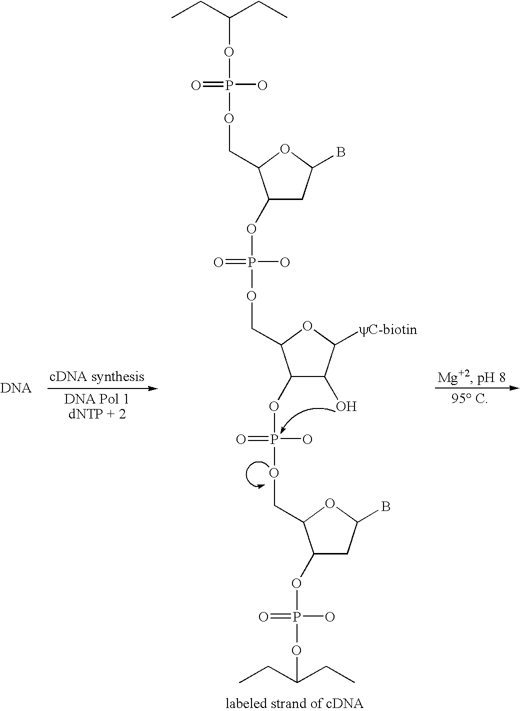

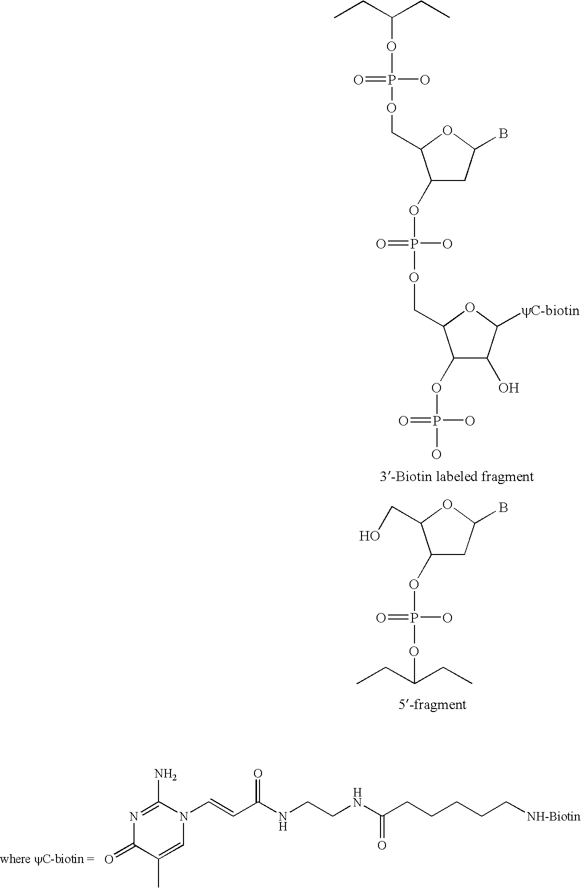

As shown above, treatment of cDNA having ribonucleotides may be cleaved by causing a transesterification reaction by treating the polymer with Mg²⁺, alkali and heat. See, e.g., Van de Sande, J. H., Loewen, P. C., and Khorana, H. G., J.Biol.Chem. Vol. 247, No. 19, pp. 6140-6148 (1972) “Studies on Polynucleotides: CXVIII, incorporated herein by reference for all purposes. A study of ribonucleotide incorporation into deoxyribonucleic acid chains by deoxyribonucleic acid polymerase I of Escherichia coli demonstrated that: “The DNA's containing CMP or GMP were selectively cleaved by alkali or specific ribonucleases and expected products were thus obtained.”

Van de Sande et al. confirmed the incorporation of ribonucleotides into DNA catalyzed by E. coli DNA polymerase I in the presence of Mn⁺⁺ with two synthetic DNA's. In general agreement with the findings of Berg et al. (1963) in Symposium on Informational Macromolecules, p. 467, Academic Press, New York), CMP and GMP could be incorporated at rates comparable to their deoxy analogs. AMP was incorporated only slowly and UMP was not incorporated at all.

Studies of the fidelity of incorporation were also conducted. Misincorporation was observed at 37° in the presence of both GTP and CTP. The misincorporation was also observed at 10° in the presence of GTP but not in the presence of CTP.

In accordance with an aspect of the present invention, ribonucleotides, e.g., 1 of the following formula may be incorporated into the growing strand of cDNA: 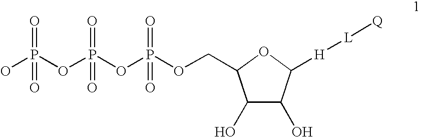
wherein H is a heterocycle, L is a linker and Q is a detectable moiety.

In many applications it is useful to directly label nucleic acid samples without having to go through amplification, transcription or other nucleic acid conversion step. This is especially true for monitoring of mRNA levels where one would like to extract total cytoplasmic RNA or poly A⁺ RNA (mRNA) from cells and incorporating labeled nucleotides in a nucleic acid polymerization step. According to one aspect of the present invention this may be accomplished by adding a labeled ribonucleotide or short labeled oligoribonucleotide to the ends of a single stranded nucleic acid. See U.S. Pat. No. 6,344,316, which is hereby incorporated by reference in its entirety for all purposes.

T4 RNA ligase catalyzes ligation of a 5′ phosphoryl-terminated nucleic acid donor to a 3′ hydroxyl-terminated nucleic acid acceptor through the formation of a 3′ to 5′ phosphodiester bond, with hydrolysis of ATP to AMP and PPi. Although the minimal acceptor must be a trinucleoside diphosphate, dinucleoside pyrophosphates (NppN) and mononucleoside 3′,5′-disphosphates (pNp) are effective donors in the intermolecular reaction. See Hoffmann and McLaughlin, Nuc. Acid. Res. 15, 5289-5303 (1987), which is hereby incorporated by reference in its entirety for all purposes.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., 3H, 125I, 35S, 14C, or 32P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, for example, a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish cites of binding of the red label from those binding the green fluorescent label. Each nucleic acid sample (target nucleic acid) can be analyzed independently from one another.

Labels can also be added to a nucleic acid sequence after fragmentation by the well known Bioting ULS labeling system. A platinum complex has two free binding sites: one to bind biotin and the other to link the complex to the purines of single stranded, double stranded, circular or linear DNA or RNA. Kits for ULS labeling are commercially available. See www.kreatech.com.

Labeling can also be done with a 2′-deoxyribonucleotide. In accordance with an aspect of the present invention, ribonucleotides, e.g., X of the following formula may be incorporated into the growing strand of cDNA: 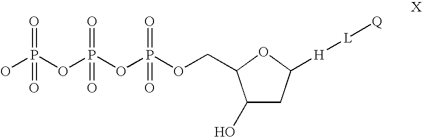
wherein H is a heterocycle, L is a linker and Q is a detectable moiety.

In many applications it is useful to directly label nucleic acid samples without having to go through amplification, transcription or other nucleic acid conversion step. This is especially true for monitoring of mRNA levels where one would like to extract total cytoplasmic RNA or poly A⁺ RNA (mRNA) from cells and incorporating labeled nucleotides in a nucleic acid polymerization step. According to one aspect of the present invention this may be accomplished by adding a labeled ribonucleotide or short labeled oligoribonucleotide to the ends of a single stranded nucleic acid. See U.S. Pat. No. 6,344,316, which is hereby incorporated by reference in its entirety for all purposes.

Hybridization

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency in this case in 6× SSPE-T at about 40° C. to about 50° C. (0.005% Triton X-100) to ensure hybridization and then subsequent washes are performed at higher stringency (e.g., 1× SSPE-T at 37° C.) to eliminate mismatched hybrid duplexes. Successive washes may be performed at increasingly higher stringency (e.g., down to as low as 0.25× SSPE-T at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide or polynucleotide probes of interest.

In a preferred embodiment, background signal is reduced by the use of a detergent (e.g., C-TAB) or a blocking reagent (e.g., sperm DNA, cot-1 DNA, etc.) during the hybridization to reduce non-specific binding. In a particularly preferred embodiment, the hybridization is performed in the presence of about 0.1 to about 0.5 mg/ml DNA (e.g., herring sperm DNA). The use of blocking agents in hybridization is well known to those of skill in the art (see, e.g., Chapter 8 in P. Tijssen, supra.)

The stability of duplexes formed between RNAs or DNAs are generally in the order of RNA:RNA>RNA:DNA>DNA:DNA, in solution. Long probes have better duplex stability with a target, but poorer mismatch discrimination than shorter probes (mismatch discrimination refers to the measured hybridization signal ratio between a perfect match probe and a single base mismatch probe). Shorter probes (e.g., 8-mers) discriminate mismatches very well, but the overall duplex stability is low.

Altered duplex stability conferred by using oligonucleotide or polynucleotide analogue probes can be ascertained by following, e.g., fluorescence signal intensity of oligonucleotide or polynucleotide analogue arrays hybridized with a target oligonucleotide or polynucleotide over time. The data allow optimization of specific hybridization conditions at, e.g., room temperature (for simplified diagnostic applications in the future).

Another way of verifying altered duplex stability is by following the signal intensity generated upon hybridization with time. Previous experiments using DNA targets and DNA chips have shown that signal intensity increases with time, and that the more stable duplexes generate higher signal intensities faster than less stable duplexes. The signals reach a plateau or “saturate” after a certain amount of time due to all of the binding sites becoming occupied. These data allow for optimization of hybridization, and determination of the best conditions at a specified temperature. Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)).

One issue that has arisen in the context of incorporating an RNA nucleotide into a cDNA is that reverse transcriptase enzymes in many cases do not incorporate ribonucleotides very well into cDNA as opposed to DNA nucleotides. Gao and Goff have seemingly solved this problem by a simple amino acid change in the Moloney Murine Leukemia Virus reverse transcriptase. The amino acid change was based in part on three dimensional structure models of the reverse transcriptase enzyme and estimations of amino acid changes which might render the Moloney enzyme capable of incorporating ribonucleotides. One successful experiment was replacement of a phenylalanine with a valine at position 155 (RT-F155V-H). Gao et al. determined that the modified enzyme was highly active in incorporating RNA triphosphates. See U.S. Pat. No. 6,136,582.

In accordance with an aspect of the present invention, it is proposed to use the mutant reverse transcriptase of Gao and Goff to incorporate the biotin labeled rNTPs as described above into cDNA transcripts. In accordance with another aspect of the present invention, as discussed above with respect to Van de Sande et al., it is also proposed that ion concentrations may be manipulated to confer the ability to incorporate ribonucleotides into DNA strands with DNA polymerase.

In accordance with an aspect of the present invention, a method is presented for analyzing a nucleic acid sample comprising RNA, having the steps of: providing a sample of RNA, e.g., mRNA; hybridizing the RNA to a primer; synthesizing cDNA with at least one labeled RNA triphosphate precursor nucleotide which is a substrate for a reverse transcriptase or a reverse transcriptase mutant to provide cDNA with a one or more biotin labeled ribonucleotides; cleaving said cDNA by initiating transesterification reactions at each site with an incorporated biotin labeled ribonucleotide by exposure of the cDNA to Mg²⁺, heat and base to provide labeled cDNA fragments; hybridizing said labeled fragments to a high density nucleic acid array to provide a hybridization pattern; and analyzing said hybridization pattern.

Preferably, the labeled RNA triphosphate precursor nucleotide has the structure 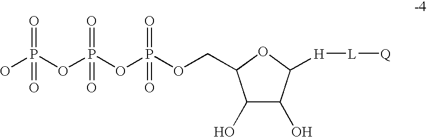
wherein H is a heterocycle, L is a linker and Q is a detectable moiety. More preferably, H is selected from the group consisting of A, G, C, U, ψ-U, ψ-iso-C. Still more preferably, H is selected from the group consisting of ψ-U and ψ-iso-C. Most preferably H is ψ-iso-C.

Preferably, Q is a detectable moiety which provides a direct signal. According to this aspect of the present invention, it is preferred that the direct signal is provided by a moiety selected from the group consisting of colloidal gold (40-80 nm diameter), fluorescein, Texas red, Rhoda mine, and green fluorescent protein.

In yet another preferred embodiment of the present invention, the detectable moiety provides an indirect signal. Preferably, this moiety is biotin which is hybridized to avidin or streptavidin having attached thereto fluorescent labels. It is also preferred that the various heterocycle moieties named above are labeled with biotin.

In a one of the most preferred aspects of the instant invention, the RNA triphosphate precursor nucleotide has the structure:

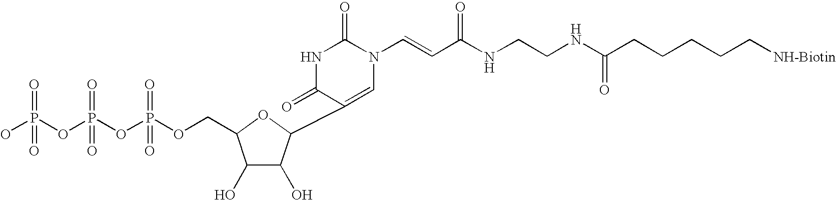

In yet another preferred embodiment of the present invention, the RNA triphosphate precursor nucleotide has the structure:

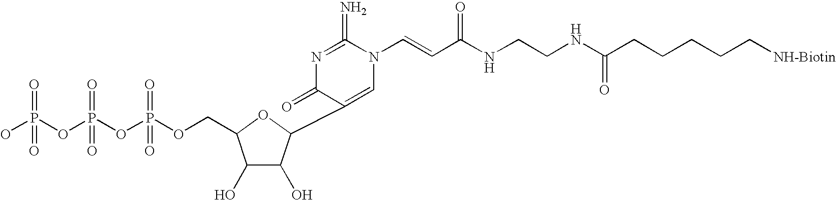

The fragment sizes range is preferably from at least 10 bps to about 200 bps. More preferably, the fragments have an average size selected from the group consisting of 10, 20, 30, 40, 50, 60, 70, 80, 100 or 200 nucleotides.

The cDNA is preferably single stranded cDNA. Alternatively, the cDNA is also preferably double stranded cDNA. Preferably, the RNA is mRNA having a poly A⁺ tail. Preferably, where the mRNA has a poly A tail, the primer has a poly dT sequence. It is also preferred that the primer has a bacterial promoter. Preferably, the promoter is selected from the group consisting of T7, SP6 and T3.

According to another aspect of the instant invention, the primers are random primers homologous to at least part of the cDNA. Preferably the random primers further comprise a promoter. The promoter is preferably selected from the group consisting of SP6, T3, and T7. Most preferably, the promoter is T7

Preferably, the reverse transcriptase or a reverse transcriptase mutant is capable of incorporating a deoxyribonucleotide, a labeled ribonucleotide and one or more deoxynucloeotides and one or more labeled ribonucleotides. Preferably, the reverse transcriptase is RT-F155V-H as de scribed U.S. Pat. No. 6,136,582.

It is understood that the examples and embodiments described herein are for illustrative purposes only and that various modifications or changes in view thereof will be suggested to persons skilled in the art and are included in the spirit and purview of this application and scope of the appended claims. All publications, patents, and patent applications cited herein are hereby incorporated by references for all purposes.

What is claimed is:

1. A method for analyzing a nucleic acid sample comprising RNA, the method comprising:
providing an RNA sample;
hybridizing said RNA to a primer;
synthesizing cDNA using a reverse transcriptase with a mixture of 2′-deoxynucleotides triphosphates, and an a labeled RNA triphosphate to provide cDNA with a plurality of labeled RNA nucleotides;
fragmenting said cDNA at each site of RNA nucleotide incorporation to provide cDNA fragments;
hybridizing said labeled fragments with a with a nucleic acid array to provide a hybridization pattern; and
analyzing said hybridization pattern.

2. A method according to claim 1 wherein said step of fragmentation is performed by transesterifying the cDNA at each site of RNA incorporation.

3. A method according to claim 2 wherein said transesterifying is caused by treatment with Mg²⁺, heat and base.

4. A method according to claim 1 wherein said labeled RNA triphosphate precursor nucleotide has the structure 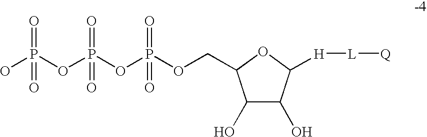
wherein H is a heterocycle, L is a linker and Q is a detectable moiety.

5. A method according to claim 4 wherein H is a synthetic base analog or a naturally occurring base variant.

6. A method according to claim 5 wherein H is selected from the group consisting of A, G, C, U, ψ-U, ψ-iso-C, 7-deazapurine, and 8-aza-7-deazapurine, 7-deazaguanosine, inosine.

7. A method according to claim 5 wherein H is selected from the group consisting of ψ-U and ψ-iso-C.

8. A method according to claim 4 wherein said labeled RNA triphosphate precursor is selected from the group consisting of

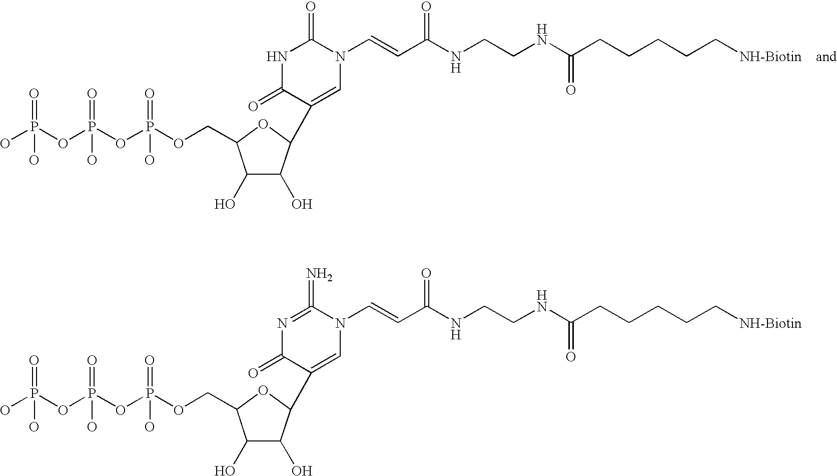

9. A method according to claim 5 wherein H is ψ-iso-C.

10. A method according to claim 4 wherein Q is a detectable moiety which provides a direct signal.

11. A method according to claim 10 wherein said direct signal is provided by the group consisting of colloidal gold (40-80 nm diameter), fluorescein, Texas red, Rhoda mine, and green fluorescent protein.

12. A method according to claim 2 wherein said detectable moiety provides an indirect signal.

13. A method according to claim 12 wherein said detectable moiety is biotin.

14. A method according to claim 1 wherein fragment sizes range from at least 10 bps to about 200 bps.

15. A method according to claim 14 wherein the fragments have an average size selected from the group consisting of 10, 20, 30, 40, 50, 60, 70, 80, 100 and 200 nucleotides.

16. A method according to claim 1 wherein said cDNA is single stranded cDNA.

17. A method according to claim 1 wherein said cDNA is double stranded cDNA.

18. A method according to claim 1 where said RNA sample is mRNA having a poly A⁺ tail.

19. A method according to claim 18 wherein said primer comprises a poly dT sequence.

20. A method according to claim 18 wherein said primers comprise random primers homologous to at least part of said cDNA.

21. A method according to claim 1 wherein said reverse transcriptase is RT-F155V-H.

22. A method according to claim 1 wherein said step of fragmentation is by fragmentation with a ribonuclease which specifically cuts at each site of incorporated RNA to provide labeled cDNA fragments.

23. A method according to claim 1 wherein said nucleic acid array is a high density nucleic acid array.

24. A method for analyzing a nucleic acid sample comprising RNA, the method comprising:
providing an RNA sample;
hybridizing said RNA to a primer;
synthesizing cDNA using a reverse transcriptase with a mixture of 2′-deoxynucleotides triphosphates, and an RNA triphosphate to provide cDNA with a plurality of incorporated RNAs;
fragmenting said cDNA at each site of RNA nucleotide incorporation to provide cDNA fragments;
labeling said fragments with a detectable label;
hybridizing said labeled fragments with a with a nucleic acid array to provide a hybridization pattern; and
analyzing said hybridization pattern.

25. A method according to claim 24 wherein said step of fragmentation is by fragmentation with a ribonuclease which specifically cuts at each site of incorporated RNA to provide labeled cDNA fragments.

26. A method according to claim 24 wherein said step of fragmentation is performed by transesterifying the cDNA at each site of RNA incorporation.

27. A method according to claim 26 wherein said transesterifying is caused by treatment with Mg²⁺, heat and base.

28. A method according to any of claims 25, 26, and 27 wherein said fragments are labeled with biotin using Biotin ULS labeling.

29. A method for analyzing a nucleic acid sample comprising RNA, the method comprising:
providing an RNA sample;
hybridizing said RNA to a primer;
synthesizing cDNA using a reverse transcriptase with a mixture of labeled and non-labeled 2′-deoxynucleotides triphosphates to provide cDNA with a plurality of labeled deoxnucleotides;
fragmenting said cDNA with DNAse I to provide cDNA fragments;
hybridizing said labeled fragments with a with a nucleic acid array to provide a hybridization pattern; and
analyzing said hybridization pattern.

30. A method according to claim 29 wherein said labeled deoxyribonucleotide has the structure 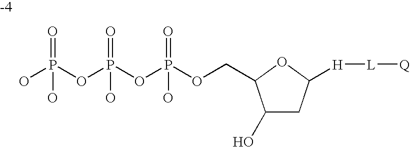
wherein H is a heterocycle, L is a linker and Q is a detectable moiety.

31. A method according to claim 30 wherein Q is biotin.
